(19) United States
(12) Patent Application Publication
(10) Pub. No.: US 2024/0398359 A1
(43) Pub. Date: Dec. 5, 2024

(54) COMBINATION OF A 2D X-RAY IMAGE RECORDING WITH A TOMOSYNTHESIS IMAGE RECORDING

(71) Applicant: Siemens Healthineers AG, Forchheim (DE)

(72) Inventors: Marcel BEISTER, Erlangen (DE); Thomas WEBER, Hausen (DE); Magdalena HERBST, Erlangen (DE); Ludwig RITSCHL, Buttenheim (DE); Julia WICKLEIN, Erlangen (DE)

(73) Assignee: Siemens Healthineers AG, Forchheim (DE)

(21) Appl. No.: 18/675,370

(22) Filed: May 28, 2024

(30) Foreign Application Priority Data
May 31, 2023 (DE) 10 2023 205 095.1

(51) Int. Cl. A61B 6/02; A61B 6/00; A61B 6/46

(52) U.S. Cl. CPC A61B 6/025; A61B 6/027; A61B 6/465; A61B 6/5223

(57) ABSTRACT

A method for generating combined X-ray image data of an examination object comprises: receiving a radiographic X-ray projection data set, which was recorded from an examination object from a first position by a radiography X-ray system; receiving a tomosynthesis X-ray projection data set, which was recorded from the examination object from a second position, which differs from the first position, by way of a tomosynthesis imaging system; generating a 2D image data set in a perspective coordinate system adapted to the geometry of the radiography X-ray system based on the radiographic X-ray projection data set; and generating a three-dimensional tomosynthesis image data set in the perspective coordinate system adapted to the geometry of the radiography X-ray system based on the tomosynthesis X-ray projection data set.

### CROSS-REFERENCE TO RELATED APPLICATION(S)

The present application claims priority under 35 U.S.C. § 119 to German Patent Application No. 10 2023 205 095.1, filed May 31, 2023, the entire contents of which is incorporated herein by reference.

### FIELD

One or more embodiments of the present invention relate to a method for generating combined X-ray image data of an examination object. In the method, a radiographic X-ray projection data set is received, which was recorded from an examination object from a first position by a radiography X-ray system. Furthermore, a tomosynthesis X-ray projection data set is received, which was recorded from the same examination object from a second position, which differs from the first position, by way of a tomosynthesis imaging system. One or more embodiments of the present invention also relate to a method for the combined X-ray image recording of an examination object. Additionally, one or more embodiments of the present invention also relate to a reconstruction facility. Moreover, one or more embodiments of the present invention relate to a combined X-ray imaging system.

### BACKGROUND

Due to physical-technical limitations, X-ray tomosynthesis recordings in a combined X-ray imaging system, which is also referred to as a radiography/fluoroscopy system, are not always possible with the same tube spacing as in normal radiography recordings. Combined image recordings, which comprise a radiography X-ray recording and a tomosynthesis X-ray recording, are also referred to as combo-mode recordings. During the reconstruction of sectional images, a perspective (as opposed to Cartesian) coordinate system is used, in which the voxel size depends upon the distance in relation to the X-ray detector and usually the central projection is used as a reference. Among other things, this offers the advantage that objects shown in focus in the sectional images are present exactly at the same pixel position and with exactly the same size as in the X-ray image of the radiography recording (1:1 pixel/voxel matching). Furthermore, this has the advantage that the “out-of-plane” artifacts occurring in the tomosynthesis due to the limited angular range do not move.

Due to the different distances between X-ray beam source and X-ray detector in the different subsystems, however, the reconstructed slices of the tomosynthesis recording no longer match the X-ray image of the radiography recording, which prevents simple navigation in the slice stack on the basis of the radiography recording, for example. The reason for this is that the voxels of the tomosynthesis recording with the same in-plane index do not lie on the projection beam or X-ray beam that was measured at the same index position in the radiography X-ray image, which is illustrated in FIG. 3.

### SUMMARY

Until now, slice images based on a tomosynthesis recording were reconstructed with either a Cartesian coordinate system or a perspective coordinate system, which is however oriented toward the central projection of the tomosynthesis or potentially also toward another true position. In both cases, it is not possible to directly read off the positions of the assigned voxels of the slice image recording on the basis of the pixel positions of the radiography recording.

There is the problem of reconstructing items of image data, which are based on a radiography recording and a tomosynthesis recording combined therewith, in a mutually consistent manner.

This object is achieved by a method for generating combined X-ray image data of an examination object according to claim 1, a method for the combined X-ray image recording of an examination object according to claim 11, a reconstruction facility according to claim 12 and a combined X-ray imaging system according to claim 13.

As already mentioned in the introduction, in the method according to an embodiment of the present invention for generating combined X-ray image data, preferably medical image data as part of a medical examination, of an examination object, a radiographic X-ray projection data set, which was recorded from an examination object from a first position by a radiography X-ray system, and a tomosynthesis X-ray projection data set, which was recorded from the examination object from a second position using a tomosynthesis imaging system in a manner of a combined X-ray system, is received. In this context, the “second position” differs from the first position. The “second position” includes a plurality of positions, from which in each case individual projection data sets were recorded to generate the tomosynthesis X-ray projection data set. These positions differ from the first position in each case. This means that the trajectory, along which an X-ray source was guided in order to record the tomosynthesis X-ray projection data set, does not include the first position. The combined X-ray system preferably has a shared X-ray beam source able to be positioned in a variable manner, which can be arranged at the first and the second position. Furthermore, the combined X-ray system preferably comprises a shared X-ray detector, which can be used to receive the X-ray radiation for generating the radiographic X-ray projection data set and the X-ray radiation for generating the tomosynthesis X-ray projection data set. Alternatively, however, the combined X-ray system can also have a first X-ray detector for receiving the X-ray radiation for generating the radiographic X-ray projection data set and a second X-ray detector for receiving the X-ray radiation for generating the tomosynthesis X-ray projection data set. The combined X-ray system can also comprise different X-ray beam sources, which are arranged at the first position or the second position. It should be noted here that the first position and, in particular, the second position can be variable. This means that the first position can be changed in different radiography recordings or can be adapted to specific requirements of a radiography recording, and the second position or a trajectory that includes the second position can likewise be adapted to specific requirements of a tomosynthesis recording.

As explained in further detail below, the radiographic X-ray projection data set can be recorded from a first distance in relation to the examination object and the tomosynthesis X-ray projection data set can be recorded from a second distance that differs from the first distance. Alternatively or additionally, the angle from which the radiographic X-ray projection data set was recorded and the angle from which the tomosynthesis X-ray projection data set was recorded can differ. In particular, an “oblique” irradiation of the X-ray radiation, which deviates from the main projection direction of the radiography recording, is also possible for the generation of the tomosynthesis X-ray projection data set.

The tomosynthesis X-ray projection data set was recorded from the same examination object from a second position, which differs from the first position, using a tomosynthesis imaging system. The radiography X-ray system and the tomosynthesis imaging system are preferably centered in relation to one another, but do not have to be centered in relation to one another, and together form the combined X-ray system. In particular, the radiography X-ray system and the tomosynthesis imaging system preferably have a shared X-ray detector However, as already mentioned, different X-ray detectors can also be configured for the radiography X-ray system and the tomosynthesis imaging system.

On the basis of the radiographic X-ray projection data set, a 2D image data set or a two-dimensional image data set is generated in a perspective coordinate system adapted to the geometry of the radiography X-ray system. In other words, the 2D image data set reproduces a projection in the z direction of the perspective coordinate system, wherein the 2D image data set is recorded at a predetermined z position, at which the X-ray detector of the combined X-ray system is located. Such a perspective coordinate system adapted to the geometry of the radiography X-ray system preferably includes a taper angle and a fan angle as well as the z-axis in the direction of the central projection direction of the radiography recording.

Furthermore, a three-dimensional tomosynthesis image data set is generated, preferably reconstructed, on the basis of the tomosynthesis X-ray projection data set.

The three-dimensional tomosynthesis image data set is generated in the perspective coordinate system adapted to the geometry of the radiography X-ray system. This means that the three-dimensional tomosynthesis image data set is generated such that, in particular, the projection areas occupied by the individual voxels at the height of the X-ray detector as part of the tomosynthesis recording match the areas of the corresponding pixels of the radiography recording. The voxels of sections of the tomosynthesis recording that lie closer toward the X-ray beam source or are at a greater distance from the X-ray detector, by contrast, have a smaller area in the plane perpendicular to the z-axis than the voxels and pixels that are assigned to the X-ray detector plane. In this manner, it is achieved that voxels of the tomosynthesis image data set have the same area coordinates as pixels of the radiography image data set that are assigned to said voxels. In this context, from a graphical perspective, the radiography image data set simply represents a projection of the tomosynthesis image data set in the direction of the X-ray beams of the conical X-ray beam of the radiography recording.

Advantageously, image regions that are localized in the radiography recording can be found at the same area coordinate position in the slice image data of the tomosynthesis X-ray projection data set, because there is no displacement between the two image data sets, as the two generated image data sets were each based on the same coordinate system.

In the method, according to an embodiment of the present invention, for the combined X-ray image recording of an examination object, a radiographic X-ray projection data set is acquired from an examination object from a first position by a radiography X-ray system.

Additionally, a tomosynthesis X-ray projection data set is acquired from the examination object from a second position, which differs from the first position, using a tomosynthesis imaging system. The radiography X-ray system and the tomosynthesis imaging system are preferably centered in relation to one another and preferably have a shared X-ray detector, but do not have to.

Finally, the method, according to an embodiment of the present invention, for generating combined X-ray image data of an examination object is applied to the acquired radiographic X-ray projection data set and the acquired tomosynthesis X-ray projection data set. The method, according to an embodiment of the present invention, for the combined X-ray image recording of an examination object advantageously delivers radiography image data sets and tomosynthesis image data sets that are coordinated with one another. In this context, for objects or regions of an image recording, the coordinates of the radiography projection match what are known as the in-plane coordinates, i.e. the area coordinates of the individual sections of the tomosynthesis recording, which span the planes perpendicular to the projection direction.

The reconstruction facility or device, according to an embodiment of the present invention, has an input interface for receiving a radiographic X-ray projection data set, which was recorded from an examination object by a radiography X-ray system from a first position, and for receiving a tomosynthesis X-ray projection data set, which was recorded from the same examination object from a second position, which differs from the first position, by way of a tomosynthesis imaging system.

The reconstruction facility or device, according to an embodiment of the present invention, also has an image generation unit for generating a 2D image data set on the basis of the radiographic X-ray projection data set in a perspective coordinate system adapted to the geometry of the radiography X-ray system. The reconstruction facility, according to an embodiment of the present invention, also has a reconstruction unit for reconstructing a three-dimensional tomosynthesis image data set on the basis of the tomosynthesis X-ray projection data set and for generating a three-dimensional tomosynthesis image data set in the perspective coordinate system adapted to the geometry of the radiography X-ray system. The reconstruction facility, according to an embodiment of the present invention, shares the advantages of the method, according to an embodiment of the present invention, for generating combined X-ray image data of an examination object.

The combined X-ray imaging system, according to an embodiment of the present invention, has a radiography X-ray system for acquiring a radiographic X-ray projection data set from an examination object from a first position. Also part of the combined X-ray imaging system, according to an embodiment of the present invention, is a tomosynthesis imaging system for acquiring a tomosynthesis X-ray projection data set from the examination object from a second position, which differs from the first position. In this context, the radiography X-ray system and the tomosynthesis imaging system are preferably centered in relation to one another, but do not have to be. The combined X-ray imaging system, according to an embodiment of the present invention, has a reconstruction facility or device according to an embodiment of the present invention. Due to the conformity of the coordinates of the voxel position of a mapped point or object of the tomosynthesis X-ray projection data set with the coordinates of the pixel position of the respective point or object in the radiographic X-ray projection data set, it is advantageously possible for the radiographic X-ray projection data set to be used for navigation in the tomosynthesis X-ray projection data set and thus image regions of interest for a diagnosis can be found in a simple and rapid manner.

A majority of the previously mentioned components of the combined imaging system and the reconstruction facility can be fully or partially implemented in the form of software modules in a processor of a corresponding computing system, for example of a control facility of the combined imaging system. An implementation largely in software has the advantage that even computing systems already in use can be easily upgraded by a software update in order to work in the manner according to an embodiment of the present invention. In this respect, the object is also achieved by a corresponding computer program product with a computer program which can be loaded directly into a computing system, with program portions for carrying out the steps of the method according to an embodiment of the present invention, at least the steps that can be executed by a computer, in particular the steps for generating a 2D image data set and for reconstructing a three-dimensional tomosynthesis image data set, when the program is executed in the computing system. Such a computer program product can comprise, where relevant, in addition to the computer program, further constituent parts such as, for example, documentation and/or additional components including hardware components, for example, hardware keys (dongles, etc.) in order to use the software.

For transport to the computing system or to the control facility and/or for storage at or in the computing system or the control facility, it is possible to use a computer-readable medium, for example, a memory stick, a hard disk or another transportable or permanently installed data carrier, on which the program portions of the computer program which can be read in and executed by a computing system are stored. For this purpose, the computing system can have, for example, one or more cooperating microprocessors or the like.

The dependent claims and the following description each contain particularly advantageous embodiments and developments of the present invention. In particular, the claims in one category of claims can also be developed in a similar way to the dependent claims in another category of claims. In addition, in the context of the present invention, the various features of different exemplary embodiments and claims can also be combined to form new exemplary embodiments.

In one variant of the method, according to an embodiment of the present invention, for generating combined X-ray image data of an examination object, the generation of a three-dimensional tomosynthesis image data set comprises the reconstruction of a three-dimensional tomosynthesis image data set on the basis of the tomosynthesis X-ray projection data set in the perspective coordinate system adapted to the geometry of the radiography X-ray system. Advantageously, a direct reconstruction of the three-dimensional tomosynthesis image data set takes place, meaning that this saves an additional step of transforming into the perspective coordinate system adapted to the geometry of the radiography X-ray system.

Alternatively, the generation of the three-dimensional tomosynthesis image data set comprises the reconstruction of a three-dimensional tomosynthesis image data set on the basis of the tomosynthesis X-ray projection data set in a perspective coordinate system that is not adapted to the geometry of the radiography X-ray system and the subsequent transformation of the three-dimensional tomosynthesis image data set into the perspective coordinate system adapted to the geometry of the radiography X-ray system. In this variant, the reconstruction of the three-dimensional tomosynthesis image data set can take place in a “familiar” coordinate system, for example a coordinate system adapted to the geometry of the recording of the tomosynthesis X-ray projection data set or a Cartesian coordinate system, in order to reduce the computing effort during the reconstruction, for example.

In one embodiment of the method, according to an embodiment of the present invention, for generating combined X-ray image data of an examination object, the tomosynthesis X-ray projection data set was recorded in such a manner that an X-ray beam source of the tomosynthesis imaging system moves along a first trajectory, which allows a recording of the examination object from different directions. Advantageously, through the variation of the position of the X-ray beam source on the first trajectory, recordings are taken of different projections from different directions, from which it is possible to obtain an item of depth information for the generation of 3D image data.

The first trajectory preferably comprises a segment of a circular path or an ellipse, the central axis of which runs orthogonally in relation to the recording direction of the radiographic X-ray projection data set. Advantageously, through the variation of the position of the X-ray beam source on the circular path or ellipse, a recording is taken of different projections from different directions, which in each case have projection beams that conform, at least in the plane that is defined by the circular path or ellipse, with projection beams for recording the 2D image data set of the radiography X-ray recording. This is due to the fact that the individual projections of the tomosynthesis X-ray projection data set were acquired from multiple different angles, due to the movement of the X-ray beam source on the circular path or ellipse. This advantageously achieves that at least voxels of the tomosynthesis image data set that are in the plane of the circular path or ellipse have the same area coordinates as pixels of the radiography image data set that are assigned to said voxels. In addition to the correct ascertaining of a depth position of a voxel in the tomosynthesis image data set, the recording of tomosynthesis projection data from different angles along the circular path achieves a reduction, at least in the plane of the circular path, of artifacts that are caused due to different directions of projection beams in the radiography projection data set and in the tomosynthesis projection data sets and the reconstruction of the image data in a shared coordinate system. The tomosynthesis recordings can be taken from different discrete angles. In this context, the movement of the X-ray beam source therefore comprises pauses, during which a recording is taken, and subsequently further movements to the next angle, during which the next recording is taken. Pauses prevent a “smudging” during the recording, caused by movement of the X-ray source, and movement artifacts caused thereby.

Alternatively, recordings are performed during a movement and a continuous change in the angle, from which the X-ray beam source irradiates the X-ray detector. By way of this approach, the duration of the entire recording process is reduced, wherein a very long recording duration in turn can lead to movements of the patient, which can likewise cause the quality of the X-ray recording to suffer.

Alternatively, the first trajectory can also comprise a straight line, which runs transversely in relation to the recording direction of the radiographic X-ray projection data set. The straight line can take place obliquely or perpendicularly in relation to the recording direction of the radiography projection data set.

Preferably, the tomosynthesis X-ray projection data set was recorded in such a manner that an X-ray beam source moves along a second trajectory, which was generated by the movement of the X-ray beam source along the first trajectory, preferably a segment of a circular path or elliptical path, being overlaid with a movement of the X-ray beam source transversely to the movement along the first trajectory. Advantageously, it is also possible to reduce artifacts in other directions than the direction, preferably the plane, which is defined by the first trajectory, preferably the circular path, for recording different projections. In particular, due to the addition of the second trajectory, the artifacts no longer have a preferential direction, which is considerably more comfortable for the observer. In addition, long thin objects that would lie in the movement direction during a one-dimensional movement are also rendered with improved localization and depth resolution.

Particularly preferably, the overlaid movement has one of the following movement types:
an additional movement, preferably a circular movement, transversely in relation to the first trajectory, preferably in relation to the circular path, the movement of the X-ray beam source,
an alternating movement transversely in relation to the first trajectory, preferably in relation to the circular path, resulting in a zig-zag movement.

In a likewise preferred manner, the first trajectory preferably comprises a circular path and the movement along the first trajectory comprises an alternating movement with a time-dependent amplitude. In this context, the movement in the transverse direction likewise comprises an alternating movement with a time-dependent amplitude, wherein the two movements are phase-shifted in relation to one another, so that a spiral movement of the X-ray beam source is generated.

Particularly preferably, the movement along the first trajectory comprises a circular path and the movement of the X-ray beam source along the first trajectory comprises an alternating movement, the amplitude of which increases as a function of the time, and the movement in the transverse direction likewise comprises an alternating movement, the amplitude of which increases as a function of the time, wherein the two movements are phase-shifted in relation to one another, so that a spiral movement is generated.

Depending on the progression and the orientation of the mapped structures, a particular movement pattern can be particularly advantageous, in order to improve the depth resolution and localization.

The 2D movements of the X-ray source during the tomosynthesis recordings along the first and optionally also the second trajectory can take place in such a manner that the individual recordings are taken during a continuous movement or, alternatively, the X-ray beam source is stopped in each case and the recordings take place while at rest in each case. As already explained, it is possible to save recording time with a continuous movement, while a recording at rest promises a simpler reconstruction or an improved image quality compared to the continuous recording, without movement artifacts.

### BRIEF DESCRIPTION OF THE DRAWINGS

The present invention is explained again below in greater detail using exemplary embodiments and with reference to the accompanying figures, in which:

FIG. 1 shows a schematic representation of an acquisition of a tomosynthesis X-ray projection data set,

FIG. 2 shows a schematic representation of an acquisition of a radiographic X-ray projection data set,

FIG. 3 shows a schematic representation of the projection beams of a combined acquisition of a tomosynthesis X-ray projection data set and a radiographic X-ray projection data set,

FIG. 4 shows a flow diagram, which illustrates a method for generating combined X-ray image data of an examination object according to an exemplary embodiment of the present invention,

FIG. 5 shows a flow diagram, which illustrates a method for the combined X-ray image recording of an examination object according to an exemplary embodiment of the present invention;

FIG. 6 shows a block diagram, which shows a reconstruction facility according to an exemplary embodiment of the present invention,

FIG. 7 shows a block diagram, which illustrates a combined X-ray imaging system according to an exemplary embodiment of the present invention.

### DETAILED DESCRIPTION

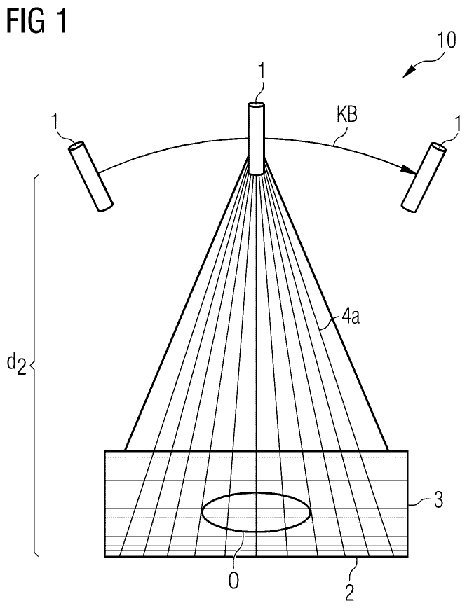

FIG. 1 illustrates a schematic representation 10 of an acquisition of a tomosynthesis X-ray projection data set. A tomosynthesis imaging system for three-dimensional X-ray image recording comprises an X-ray beam source 1, which can be rotated about an object center point and which is located at a predetermined distance d2 from an X-ray detector 2 of the tomosynthesis imaging system and with which X-ray image recordings of an examination object O can be performed from various directions or angles. To this end, the X-ray beam source 1 is moved over a circular path KB and individual images of the examination object O are generated from different angles relative to the central projection axis of the tomosynthesis imaging system. A three-dimensional slice image is calculated from the individual images, which maps a large number of slices 3 of the examination object O. An X-ray beam 4a is transmitted in the form of a fan beam or a cone beam from the X-ray beam source 1, through the examination object O, and is detected by the X-ray detector 2.

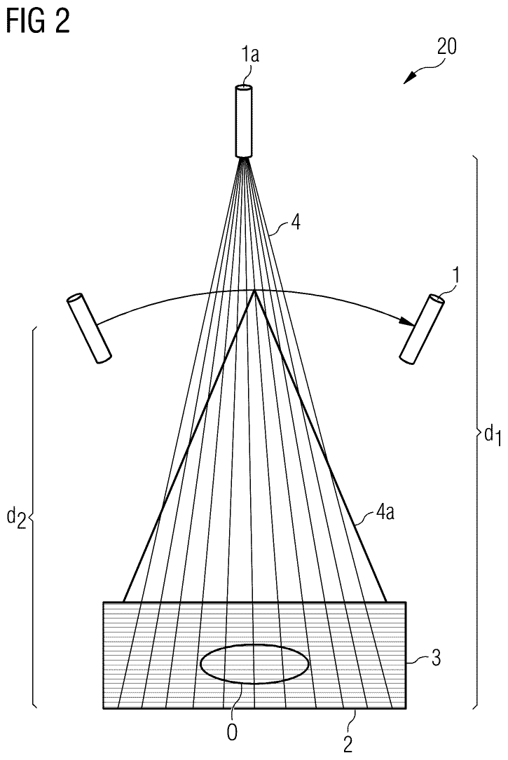

FIG. 2 shows a schematic representation 20 of a combined acquisition of a radiographic X-ray projection data set and a tomosynthesis projection data set. A radiography X-ray system likewise comprises an X-ray beam source 1a, which is located at a distance d1 from the X-ray detector 2 that is shared with the tomosynthesis imaging system.

The tomosynthesis imaging system for three-dimensional X-ray image recording already explained in FIG. 1, which in the arrangement shown in FIG. 2 is now combined with the radiography X-ray system, is shown in FIG. 2. The tomosynthesis imaging system comprises an X-ray beam source 1, which can be rotated about an object center point and which is located at a predetermined distance d2 from an X-ray detector 2 of the tomosynthesis imaging system and with which X-ray image recordings of an examination object O can be performed from various directions or angles.

The image data generated on the basis of the radiographic X-ray projection data set can be used for navigation in the slice stack or stack of slices 3 generated on the basis of the tomosynthesis projection data set, if the area coordinates of the radiography projection or the image data based thereon can be transferred to the slices 3 of the tomosynthesis image data.

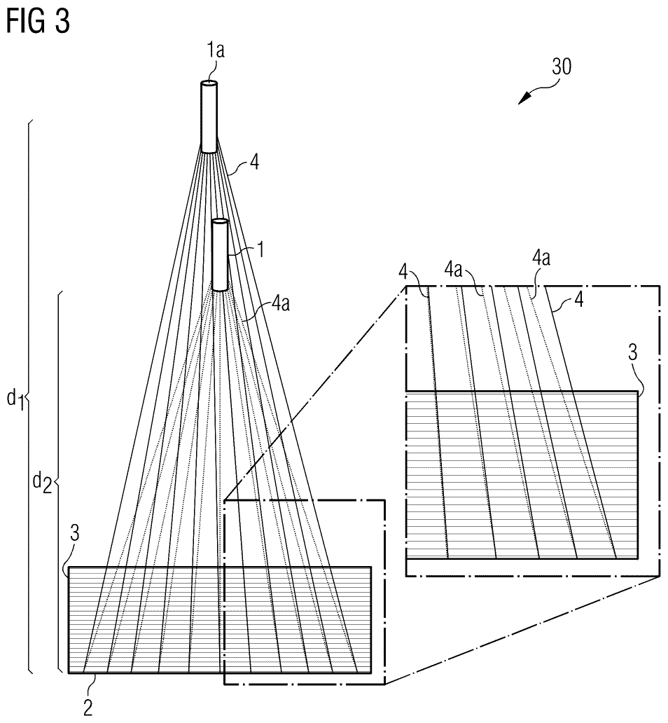

FIG. 3 illustrates a schematic representation 30 of the projection beams or X-ray beams 4a, 4 of a combined acquisition of a tomosynthesis X-ray projection data set and a radiographic X-ray projection data set. As shown in an enlarged representation on the right in FIG. 3, depending on the angular position of the X-ray beam source 1 of the tomosynthesis imaging system, a different X-ray beam or projection beam 4a of the X-ray beam source 1 of the tomosynthesis imaging system runs in a conforming manner with an X-ray beam or projection beam 4 of the X-ray source 1a of the radiography X-ray system. Since the tomosynthesis X-ray projection data sets are in each case generated by applying projection beams that conform with a projection beam of the radiography X-ray system, the reconstruction can take place without artifacts directly in the coordinate system of the radiography X-ray system. A movement of the X-ray beam source 1 of the tomosynthesis system now also takes place transversely in relation to the plane of the sheet, thus the projections of the tomosynthesis also comprise image data sets with projection beams that conform with projection beams of the radiography X-ray projection data set that run outside of the plane of the sheet, meaning that a tomosynthesis slice image data set can also be reconstructed without artifacts in the direction perpendicular to the plane of the sheet.

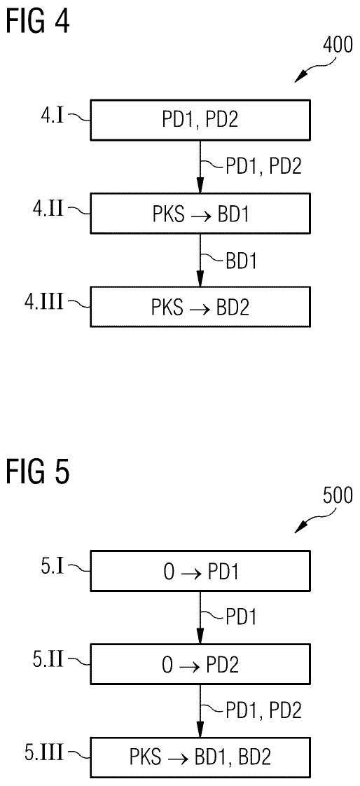

FIG. 4 shows a flow diagram 400, which illustrates a method for generating combined X-ray image data of an examination object according to an exemplary embodiment of the present invention.

In step 4.I, first a radiographic X-ray projection data set PD1 and a tomosynthesis X-ray projection data set PD2 are received.

In step 4.II, a 2D image data set BD1 is reconstructed in a perspective coordinate system PKS adapted to the geometry of the radiography X-ray system on the basis of the radiographic X-ray projection data set PD1.

In step 4.III, a three-dimensional tomosynthesis image data set BD2 is reconstructed in the perspective coordinate system PKS adapted to the geometry of the radiography X-ray system on the basis of the tomosynthesis X-ray projection data set PD2.

FIG. 5 illustrates a flow diagram, which shows a method for the combined X-ray image recording of an examination object according to an exemplary embodiment of the present invention.

In step 5.I, a radiographic X-ray projection data set PD1 is acquired from an examination object O.

In step 5.II, a tomosynthesis projection data set PD2 is acquired from an examination object O.

In step 5. III, the method for generating combined X-ray image data of an examination object illustrated in FIG. 4 is performed.

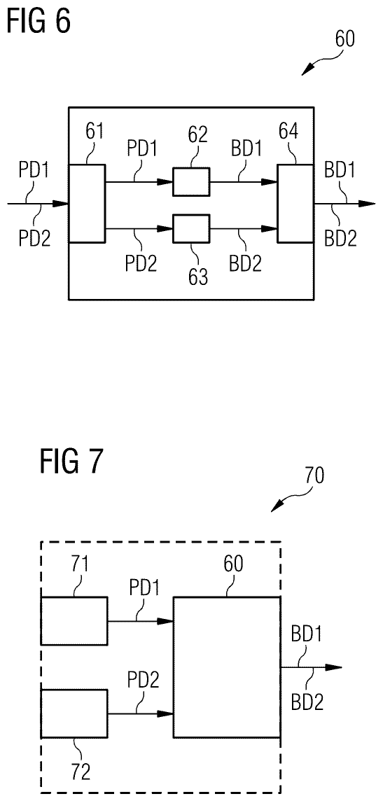

FIG. 6 illustrates a block diagram, which shows a reconstruction facility 60 according to an exemplary embodiment of the present invention.

The reconstruction facility 60 has an input interface 61. The input interface 61 is configured to receive a radiographic X-ray projection data set PD1, which was recorded from a first distance d1 from an examination object O by a radiography X-ray system, and to receive a tomosynthesis X-ray projection data set PD2, which was recorded from the same examination object O from a second distance d2, which differs from the first distance d1, using a tomosynthesis imaging system.

An image generation unit 62 for generating a 2D image data set BD1 on the basis of the radiographic X-ray projection data set PD1 in a perspective coordinate system adapted to the geometry of the radiography X-ray system is also part of the reconstruction facility 60.

The reconstruction facility 60 also comprises a reconstruction unit 63 for reconstructing a three-dimensional tomosynthesis image data set BD2 in the perspective coordinate system PKS adapted to the geometry of the radiography X-ray system on the basis of the tomosynthesis X-ray projection data set PD2.

An output interface 64 for outputting the generated image data sets BD1, BD2 is also part of the reconstruction facility 60.

FIG. 7 shows a combined X-ray imaging system 70. The combined X-ray imaging system 70 comprises a radiography X-ray system 71 for acquiring a radiographic X-ray projection data set PD1 from an examination object O (not shown) from a first distance d1. Furthermore, the combined X-ray imaging system 70 comprises a tomosynthesis imaging system 72 for acquiring a tomosynthesis X-ray projection data set PD2 from the examination object O from a second distance d2, which differs from the first distance d1, wherein the radiography X-ray system 71 and the tomosynthesis imaging system 72 are centered in relation to one another. A reconstruction facility 60, as illustrated in FIG. 6, is also part of the combined X-ray imaging system 70.

Finally, it should again be noted that the methods and apparatuses described above are merely preferred exemplary embodiments of the present invention and that the present invention can be modified by a person skilled in the art without departing from the field of the present invention, to the extent that it is specified by the claims. Thus, the two methods, the image reconstruction facility and the combined X-ray imaging system have been described primarily on the basis of a system for recording medical image data. However, the present invention not restricted to application in the medical field, but instead the present invention can also be applied in principle to the recording of images for other purposes. For the sake of completeness, it should be noted that the use of the indefinite articles “a” or “an” does not preclude the relevant features from also being present in plural. Likewise, the term “unit” does not exclude the possibility that said unit consists of a plurality of components, which may also be spatially distributed if applicable.

Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the present invention has been shown and described with respect to certain example embodiments, equivalents and modifications will occur to others skilled in the art upon the reading and understanding of the specification. The present invention includes all such equivalents and modifications and is limited only by the scope of the appended claims.

What is claimed is:

1. A method for generating combined X-ray image data of an examination object, the method comprising:
receiving a radiographic X-ray projection data set recorded from an examination object from a first position by a radiography X-ray system;
receiving a tomosynthesis X-ray projection data set recorded from the examination object from a second position by way of a tomosynthesis imaging system, the second position being different from the first position;
generating, based on the radiographic X-ray projection data set, a 2D image data set in a perspective coordinate system adapted to a geometry of the radiography X-ray system; and
generating, based on the tomosynthesis X-ray projection data set, a three-dimensional tomosynthesis image data set in the perspective coordinate system adapted to the geometry of the radiography X-ray system.

2. The method as claimed in claim 1, wherein
the first position includes a first distance from which the radiographic X-ray projection data set was recorded,
the second position includes a second distance from which the tomosynthesis X-ray projection data set was recorded, and
the first distance and the second distance differ from one another.

3. The method as claimed in claim 1, wherein the radiography X-ray system and the tomosynthesis imaging system are centered relative to one another.

4. The method as claimed in claim 1, wherein the generating of a three-dimensional tomosynthesis image data set comprises:
reconstructing, based on the tomosynthesis X-ray projection data set, the three-dimensional tomosynthesis image data set in the perspective coordinate system adapted to the geometry of the radiography X-ray system.

5. The method as claimed in claim 1, wherein the generating of a three-dimensional tomosynthesis image data set comprises:
reconstructing the three-dimensional tomosynthesis image data set in a perspective coordinate system that is not adapted to the geometry of the radiography X-ray system based on the tomosynthesis X-ray projection data set and a subsequent transformation of the three-dimensional tomosynthesis image data set into the perspective coordinate system adapted to the geometry of the radiography X-ray system.

6. The method as claimed in claim 1, wherein the tomosynthesis X-ray projection data set is recorded by moving an X-ray beam source of the tomosynthesis imaging system along a first trajectory to record the tomosynthesis X-ray projection data set from different directions, so that the tomosynthesis X-ray projection data set includes a plurality of X-ray projection data sets acquired from multiple different angles due to the movement of the X-ray beam source along the first trajectory.

7. The method as claimed in claim 6, wherein the first trajectory comprises one of the following trajectory types:
a segment of a circular path or an ellipse, a central axis of which runs orthogonally relative to a recording direction of the radiographic X-ray projection data set, or
a line, which runs transversely relative to the recording direction of the radiographic X-ray projection data set.

8. The method as claimed in claim 6, wherein the tomosynthesis X-ray projection data set is recorded by moving an X-ray beam source along a second trajectory, which is generated by the movement of the X-ray beam source along the first trajectory being overlaid with the movement of the X-ray beam source transversely to the movement along the first trajectory.

9. The method as claimed in claim 8, wherein the overlaid movement has one of the following movement types:
an additional movement transversely relative to the first trajectory, or
an alternating movement transversely relative to the first trajectory, resulting in a zig-zag movement.

10. The method as claimed in claim 9, wherein
the movement along the first trajectory includes an alternating movement with a time-dependent amplitude,
the movement in a transverse direction includes an alternating movement with a time-dependent amplitude, and
the movement along the first trajectory and the movement in the transverse direction are phase-shifted relative to one another to generate a spiral movement of the X-ray beam source.

11. A method for the combined X-ray image recording of an examination object, the method comprising:
acquiring a radiographic X-ray projection data set from an examination object from a first position by way of a radiography X-ray system;
acquiring a tomosynthesis X-ray projection data set from the examination object from a second position by way of a tomosynthesis imaging system, the second position differing from the first position; and
performing the method as claimed claim 1.

12. A reconstruction device, comprising:
an input interface configured to
receive a radiographic X-ray projection data set recorded from an examination object from a first position by a radiography X-ray system, and
receive a tomosynthesis X-ray projection data set recorded from the examination object from a second position by way of a tomosynthesis imaging system, the second position differing from the first position;
an image generation unit configured to generate, based on the radiographic X-ray projection data set, a 2D image data set in a perspective coordinate system adapted to a geometry of the radiography X-ray system; and
a reconstruction unit configured to generate, based on the tomosynthesis X-ray projection data set, a three-dimensional tomosynthesis image data set in the perspective coordinate system adapted to the geometry of the radiography X-ray system.

13. A combined X-ray imaging system, comprising:
a radiography X-ray system configured to acquire a radiographic X-ray projection data set from an examination object from a first position;
a tomosynthesis imaging system configured to acquire a tomosynthesis X-ray projection data set from the examination object from a second position, the second position differing from the first position; and
a reconstruction device as claimed in claim 12.

14. A non-transitory computer program product comprising commands that, when executed by at least one processor at a reconstruction device, cause the reconstruction device to perform the method of claim 1.

15. A non-transitory computer-readable medium comprising computer-readable instructions that, when executed by at least one processor at a reconstruction device, cause the reconstruction device to perform the method of claim 1.

16. The method as claimed in claim 2, wherein the radiography X-ray system and the tomosynthesis imaging system are centered relative to one another.

17. The method as claimed in claim 2, wherein the generating of a three-dimensional tomosynthesis image data set comprises:
reconstructing the three-dimensional tomosynthesis image data set in a perspective coordinate system that is not adapted to the geometry of the radiography X-ray system based on the tomosynthesis X-ray projection data set and a subsequent transformation of the three-dimensional tomosynthesis image data set into the perspective coordinate system adapted to the geometry of the radiography X-ray system.

18. The method as claimed in claim 17, wherein the tomosynthesis X-ray projection data set is recorded by moving an X-ray beam source of the tomosynthesis imaging system along a first trajectory to record the tomosynthesis X-ray projection data set from different directions, so that the tomosynthesis X-ray projection data set includes a plurality of X-ray projection data sets acquired from multiple different angles due to the movement of the X-ray beam source along the first trajectory.

19. The method as claimed in claim 5, wherein the tomosynthesis X-ray projection data set is recorded by moving an X-ray beam source of the tomosynthesis imaging system along a first trajectory to record the tomosynthesis X-ray projection data set from different directions, so that the tomosynthesis X-ray projection data set includes a plurality of X-ray projection data sets acquired from multiple different angles due to the movement of the X-ray beam source along the first trajectory.

20. The method as claimed in claim 7, wherein the tomosynthesis X-ray projection data set is recorded by moving an X-ray beam source along a second trajectory, which is generated by the movement of the X-ray beam source along the first trajectory being overlaid with the movement of the X-ray beam source transversely to the movement along the first trajectory.
